(19) United States
(12) Patent Application Publication
(10) Pub. No.: US 2012/0034628 A1
(43) Pub. Date: Feb. 9, 2012

(54) STANDARDIZED EVALUATION OF THERAPEUTIC EFFICACY BASED ON CELLULAR BIOMARKERS

(75) Inventors: Stephen A. Lesko, Baltimore, MD (US); Paul O.P. Ts'o, Ellicott, MD (US)

(73) Assignee: On-Q-Ity Inc., Waltham, MA (US)

(21) Appl. No.: 13/278,873

(22) Filed: Oct. 21, 2011

Related U.S. Application Data
(63) Continuation of application No. 12/589,510, filed on Jun. 10, 2009.
Application No. 12/589,510 is a continuation of application No. 10/787,585, filed on Feb. 27, 2004, now abandoned.
(60) Provisional application No. 60/451,050, filed on Feb. 27, 2003.
(60) Provisional application No. 60/488,865, filed on Jul. 21, 2003.

(51) Int. Cl. G01N 33/566

(52) U.S. Cl. 435/7.23; 435/7.1

(57) ABSTRACT

The present invention provides materials and methods for predicting the response of a disease state to a therapeutic agent. A targeting moiety specific for a biological marker is labeled with a reporter moiety and used to analyze cells characteristic of the disease state. The output of the reporter moiety, which may be fluorescence intensity, is compared to the output of reference standard analyzed under similar or identical conditions. The use of a reference standard allows biomarker reporting to be normalized. Biomarker values can then be correlated from sample to sample and from laboratory to laboratory based on quantitative calibration on a universal reference standard.

### CROSS REFERENCE TO RELATED APPLICATIONS

This application is a continuation of U.S. patent application Ser. No. 12/589,510, filed Jun. 10, 2009, which is a continuation of U.S. patent application Ser. No. 10/787,585, filed Feb. 27, 2004 (now abandoned), which claims the benefit of U.S. Provisional Application Nos. 60/451,050, filed Feb. 27, 2003 and 60/488,865, filed Jul. 21, 2003, the contents of each are specifically incorporated herein by reference in their entirety.

### FIELD OF THE INVENTION

The present invention relates in general to pharmaceutical therapies, and more particularly to compounds and methods for predicting the efficacy of particular therapies for particular patients.

### BACKGROUND

One of the most urgent needs for cancer patients is to find an effective drug, particularly for cancer patients with a metastatic disease after removal/destruction of the primary tumor in the original organ site by surgery or radiation. The goal of any therapy is to improve the life expectancy and quality of life in a cost-effective manner. Through much effort and expense, a number of new anticancer drugs have been developed and approved in the past decade for use in clinics. These recently available drugs are aimed toward different cellular targets based on different mechanisms. However, the use of these drugs so far has been empirical, and not based on diagnostic drug-action response parameters, with overall modest benefit, approximately 20-30% favorable responses. In addition, the only available criteria for evaluating a response have been based upon imaging/radiographic measurements or observable clinical changes during or after 2-3 months of this empirical therapy. There is potentially a significant opportunity loss for therapy if the disease is still progressing (70-80% of the cases) with added toxic side effects to all the patients.

Different patients respond differently to specific therapies, and (especially in cases where the therapy has undesirable side effects) an important element of treatment is selection of the appropriate therapy. Even in cases where side effects are minimal, it is desirable to avoid selection of expensive, but ineffective, therapies.

Many therapies have predictable efficacy for specific patients which may be determined based on whether or not particular cells (which may be obtained from the patient's tissue, cells circulating in a bodily fluid or individual cells) have certain markers (referred to herein as biomarkers) which are specific to the disease and/or the proposed treatment. There are a variety of techniques for identifying the presence of biomarkers on cells. Typically, a moiety with an affinity for the biomarker (for example, an antibody, DNA, RNA, oligonucleotide, receptor-specific ligand or the drug under investigation itself) is coupled to a reporting moiety (for example, a fluorescent dye, a magnetic bead, a radioactive compound or an enzyme) and the resulting compound brought in contact with a group of cells of interest. The mixture is then processed so as to quantify the number or a ratio of cells tagged by the reporting moiety.

Cells may be obtained from solid tissue biopsy. Alternatively, they may be isolated from body fluids (see, for example, U.S. Pat. No. 5,962,237). One example of a commercially available method for the isolation of circulating cancer cells is the Circulating Cancer Cell or Blood Biopsy™ test (Cell Works Inc., Baltimore Md.). This test is designed to enrich and identify intact cancer cells from blood with the following protocol: 1) enrich cancer cells from 15-20 ml blood using double-gradient centrifugation and immunomagnetic beads to remove blood cells (negative selection); 2) deposit remaining cells on a microscope slide and stain with an antibody cocktail (e.g., FITC-labeled antibodies with reactivity to nine cytokeratin (CK) peptides and a tumor-associated glycoprotein expressed on human carcinomas) and a DNA specific reagent (DAPI); 3) scan slides with a fluorescence microscope and acquire digital images of FITC-positive cells. Recovery studies were done with 2-3 cancer cell lines from a number of cancers (breast, colon, gastric, liver, lung, pancreas, prostate) by quantitatively spiking cancer cells into blood (10-100 cancer cells in 20 ml blood sample, 6 replicates). All cell lines showed good quality staining (100% of the cells were positively stained). A mean recovery of 55 to 85% was found across 19 cell lines. A typical within-run average recovery ranged from 63% to 78% (SD=5% to 15%) and within-run CV % from 8% to 19%. The circulating cancer cell test has been applied to about 60 non-spiked, blood samples from normal controls and a positively stained cell has never been detected.

Although many therapies benefit from pre-screening patients to identify likely responders, one example is trastuzumab where a patient with breast cancer is routinely tested for the HER2/neu receptor prior to treatment, in order to determine whether the therapy is appropriate. The HER2/neu gene is overexpressed or amplified in approximately 20 to 25% of human breast cancers. Trastuzumab is a very effective therapy, but it has undesirable side effects and only 30 to 35% of selected breast cancer patients respond to trastuzumab as a single agent. To qualify for and receive benefit from this therapy, patients must have tumors that overexpress the HER-2 protein, most commonly measured by fluorescence in situ hybridization (FISH) or immunohistochemistry (IHC). Since the protein is present in both normal and abnormal cells, it is necessary to measure the degree of overexpression, not just the presence of HER-2 in the cell. Current IHC analysis relies on a subjective interpretation by the pathologist using solely a microscope and the human eye, and characterization into one of four groups: in the case of HERCEPTEST, a test for Her-2 overexpression in a breast cancer tissue, by scoring from 0 to 3+, with samples scoring 3+ regarded as positive, samples scoring 0 or 1+ as negative, and the remaining scores as requiring other testing. In an effort to reduce the subjective human factor, a similar test is provided by ACIS (available from ChromaVision, San Juan Capistrano, Calif.), which uses an image analyzer to attempt to quantify staining; scores from 0 to 4 are generated, with a score less than 2 considered negative, a score greater than 2 considered positive, but with alternate testing recommended for scores between 0.5 and 1.9. Data on the clinical validity of such characterizations are, however, inconclusive, possibly because of the subjective nature of the determination, variability of test conditions, variability of different scorers' techniques or variability among laboratory equipment. See, for example, Formier et al., HER2 Testing and Correlation with Efficacy of Trastuzumab Therapy, Oncology Vol 16 No. 10 p 1340, incorporated herein by reference.

Because of the cost of therapeutic agents and undesirable side effects (and the risks of time lost pursuing an ineffective therapy), a standardized, quantitative method for characterizing a patient's predicted responsiveness to treatment would be highly desirable.

### SUMMARY OF THE INVENTION

The foregoing problems are overcome, and other advantages are provided by an objective approach to a standardized quantification of test results. It is an object of the invention to provide a universally standardized, quantified, measure of the presence of biomarkers in a cell.

In one aspect, the present invention provides a method for predicting a patient's response to a specific proposed pharmaceutical therapy. Methods of the invention may comprise selecting a proposed therapy, determining a type of cell which is a target for said proposed therapy, and determining a biomarker associated with said cell, the presence, absence, and/or amount of said biomarker being indicative of the likelihood of said patient's response to said proposed therapy. In some aspects, methods of the invention may comprise identifying a targeting moiety having an affinity for said biomarker. In some embodiments, a targeting moiety may be the same or different as a therapeutic agent to be used in the therapy. In other aspects, the targeting moiety may interact with the same biomarker or cellular target as the therapeutic agent but be different from the therapeutic agent. In one aspect, a reporter moiety compatible with said targeting moiety may be selected and coupled to the targeting moiety. For example, a therapeutic agent that interacts with a specific biomarker may be coupled with a reporter moiety. Those skilled in the art will appreciate that coupling said targeting moiety with said reporter moiety may be performed in such a manner that the properties of each are unaffected. Such a targeting moiety (e.g., therapeutic agent) coupled to a reporting moiety may be used as a test compound. A sample containing said a cell to be tested may be reacted with a test compound so as to create a processed sample. A processed sample may be evaluated, for example, comparing the intensity of fluorescence observed to that of a reference standard. This may be accomplished, for example, by calibrating a test instrument by creating a plot of intensity against exposure time, selecting a linear range of said plot arid selecting as a standard exposure the exposure time which produces approximately the same intensity measurement on each other test instrument, obtaining a digital image for the processed sample at said standard exposure, and determining a density representing the amount of biomarker expressed as a fraction of the intensity exhibited by a reference standard at an equivalent exposure. A reference standard may be any compound or material that produces a reproducible signal. One example of a reference standard that may be used as a calibrating reagent is a fluorescent microbead.

In another aspect, the present invention provides a method of selecting a therapeutic agent for the treatment of cancer. Such a method may comprise obtaining a cell sample from a patient, wherein the sample comprises circulating cancer cells. Typically, said cancer cells may comprise one or more biomarkers. Cancer cells may then be contacted with a test compound (e.g., a targeting moiety coupled to a reporter moiety) that specifically binds to one or more biomarker. Test compounds typically comprise one or more reporter moieties, for example, a fluorescent moiety. The intensity of the fluorescence of the cells may be measured, for example, using a fluorescence microscope; and the intensity of the fluorescence of the cells may be compared to that of a reference standard. The fluorescent intensity of the cells is typically measured under standardized conditions (e.g., at the same time of exposure). Typically, the ratio of the intensity of the stained cells to the reference standard correlates to the effectiveness of the therapeutic agent against the cancer. The ratio may be conveniently expressed as a percent of the reference standard. For example, the presence and/or increased amount of a biomarker (indicated by increased fluorescence) may correlate to susceptibility of the cancer cell to the therapeutic reagent. Likewise the presence and/or increase amount of a particular biomarker may indicate resistance to the therapeutic. The absence and/or reduced amount of a particular biomarker may indicate susceptibility of the cancer to the therapeutic or the absence and/or reduced amount may indicate resistance to the therapeutic. Those skilled in the art will appreciate that the amount of fluorescence may be compared to a reference standard, for example, a known cancer cell of known type and susceptibility.

The present invention also provides kits for the practice of one or more methods of the invention. For example, the present invention provides a kit for determining the susceptibility of a cancer cell to a therapeutic agent, comprising a targeting moiety specific for a biomarker. A targeting moiety may be the therapeutic agent. A targeting moiety may be coupled to a fluorescent moiety. One skilled in the art will appreciate that a targeting moiety (e.g., a therapeutic agent) may be of any type known in the art, for example, small molecules, peptides, proteins, enzymes, monoclonal antibodies, oligonucleotides (DNA, RNA, mixed DNA and RNA, which may contain one or more non-naturally occurring nucleotide) and the like. Kits of the invention may comprise one or more therapeutic agents, which may be coupled to one or more fluorescent moieties. A targeting moiety and/or a therapeutic agent may comprise one or more antibodies, which may include one or more monoclonal antibodies. Kits of the invention may comprise at least one antibody, which may be a monoclonal antibody, which is specific for a cytokeratin. Antibodies of the kits of the invention may be polyclonal or monoclonal antibodies and may comprise a fluorescent moiety. Kits of the invention may comprise a reference standard. Such reference standards may comprise a known amount of a non-bleaching fluorescent moiety. One example of a suitable reference standard is a fluorescent microsphere. Kits of the invention may comprise one or more reagent selected from the group consisting of buffers, buffer salts, detergents, surfactants, fixatives, and the like. In a particular embodiment, kits of the invention may comprise a permeability buffer.

### BRIEF DESCRIPTION OF THE FIGURES

FIG. 1 is a standard curve of observed fluorescence per pixel plotted against time of exposure.

FIG. 2 is a standard curve of observed fluorescence per pixel plotted against time of exposure.

FIG. 3 is a standard curve of observed fluorescence per pixel plotted against time of exposure.

FIG. 4 is a standard curve of observed fluorescence per pixel plotted against time of exposure.

### DESCRIPTION OF THE PREFERRED EMBODIMENTS

In general, the present invention provides a method of selecting a therapeutic agent for treatment of a disease state. The selection may be performed by analyzing a cell that that is characteristic of the disease state (e.g., a cancer cell), for the presence, absence and/or amount of one or more biomarkers. The analysis may be accomplished by contacting the cell with a targeting moiety that specifically interacts (e.g., binds to) a biomarker characteristic of the disease state. The presence, absence and/or amount of one or more biomarkers may quantified (e.g., as a fluorescence intensity) and compared to a reference standard. By comparison to a reference standard, the present invention allows comparison of results obtained at different times, from different laboratories, and/or from different patients. The present invention is particularly useful in monitoring the time course of a disease state after initiation of a treatment regimen.

The characterization of isolated, individual cancer cells in circulation is different from the characterization of cancer cells in tissue sections from a tumor biopsy. Cancer cells in tissue sections are embedded in groups of cells which usually have a distinct orientation pattern and morphological characteristics (both cellular and nuclear morphology) readily recognized under the microscope by experienced pathologists, since the normal cells are in juxtaposition to the cancer cells for comparison. Thus, the morphology and orientation in groups provide the criteria for distinction between normal and cancerous tissues. For isolated individual cancer cells in circulation, the group morphology and orientation pattern are not available as references. Therefore, the study of the individual cancer cells in circulation must be based on quantitative biomarker measurement of individual cells.

On the other hand, circulating cancer cells are individual cells, and these individual entities can be stained (equally accessible by the stain) and measured optically much more uniformly and quantitatively, particularly by monoclonal antibodies attached to fluorescent dyes. The measurement is on a one to one equivalent basis and not based on chemical reactions for amplification. Chemical/enzymatic reaction for producing colored products in amplification of the optical signals is very effective in signal enhancement, but it may be very difficult to control the reaction to measure the color quantitatively and reproducibly from slide to slide and from sample to sample. Tissue sections are not usually of a uniform one-cell thickness; typically, the cell layers overlap each other. It would require very accurate optical focusing to select an optical plane comprising an entire cell and it may not be possible to have one horizontal, level optical plane consisting of only one cell. The boundary of the cells is variable and not so easily recognizable in tissue sections. For all these reasons, having a quantitative measurement of the optical signal from cancer cells in a tissue section will be much more difficult than having one from single cancer cells in circulation. The ease of identifying cell boundaries makes the methods of the present invention particularly well-suited for semiautomatic, computerized procedures.

Examples are given here on how biomarkers relevant to selection of therapeutic treatment can be measured for cultured breast cancer cells spiked in a blood sample. In particular, the fluorescently labeled trastuzumab is used to measure quantitatively the trastuzumab receptor (HER-2/neu biomarker) in a quantitative, numerical manner based on a universal standard of reference. Other suitable combinations of biomarkers and therapeutic agents include, but are not limited to, those listed in the following table.

| Biomarker | Therapeutic agent |
|---|---|
| Ribonuclease Reductase | Gemcitabine |
| ERCC-1 (Excision Repair Cross | Cisplatinum |
| Complementary 1) | |
| β Tubulin III | Paclitaxel and Vinorelbine |
| | (Vinca Alkaloid) |
| Thymidylate Synthase | 5 FU-related drugs |
| ErbB1/EGFR | gefitinib or cetuximab |
| ErbB2/HER-2/neu | trastuzumab |
| Vascular endothelial cell growth factor | bevacizumab |

The approach can be illustrated by outlining a method utilizing a fluorescently labeled specific antibody that yields a quantitative value for the HER-2 protein as related to a non-bleaching fluorescence reference standard. In this way, digital values can be compared from sample to sample and from laboratory to laboratory based on a quantitative calibration. One skilled in the art will readily appreciate that any targeting moiety, which may be coupled to any reporter moiety, that specifically interacts with a biomarker may be used in the practice of the invention. Suitable examples of targeting moieties include, but are not limited to, small molecules, peptides, proteins, enzymes, monoclonal antibodies, oligonucleotides (DNA, RNA, mixed DNA and RNA, which may contain one or more non-naturally occurring nucleotide) and the like.

It will be readily apparent to those of ordinary skill in the relevant arts that other suitable modifications and adaptations to the methods and applications described herein are obvious and can be made without departing from the scope of the invention or any embodiment thereof. Having now described the present invention in detail, the same will be more clearly understood by reference to the following examples, which are included herewith for purposes of illustration only and are not intended to be limiting of the invention.

### Example 1

Initially, an attempt to obtain the desired results using the obvious approach, and the failure of that approach, will be described.

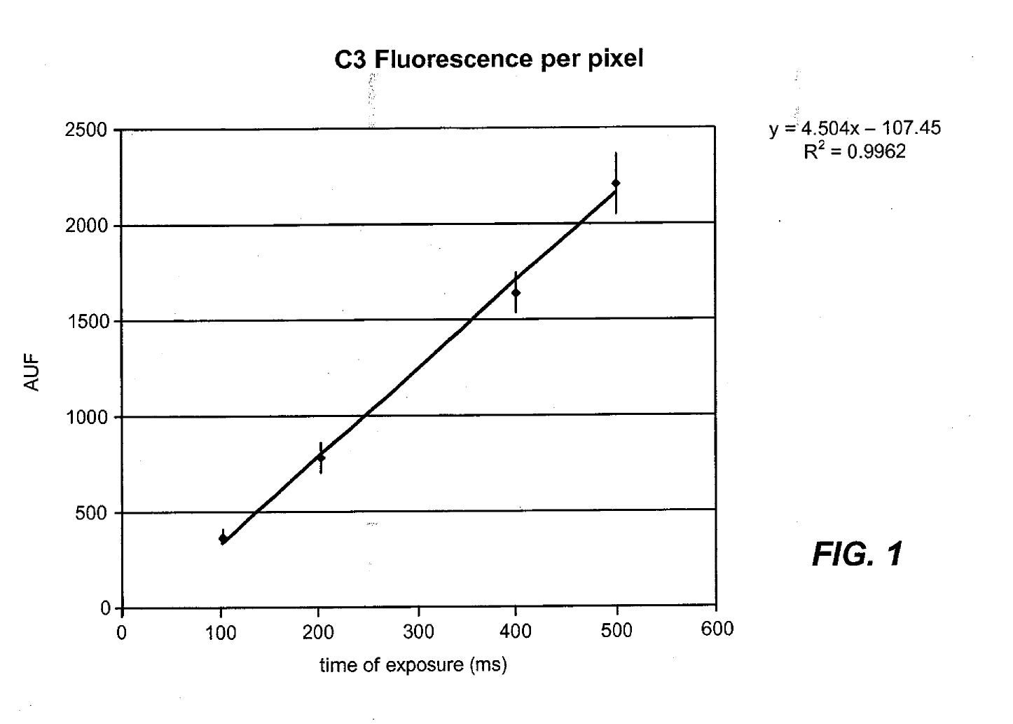

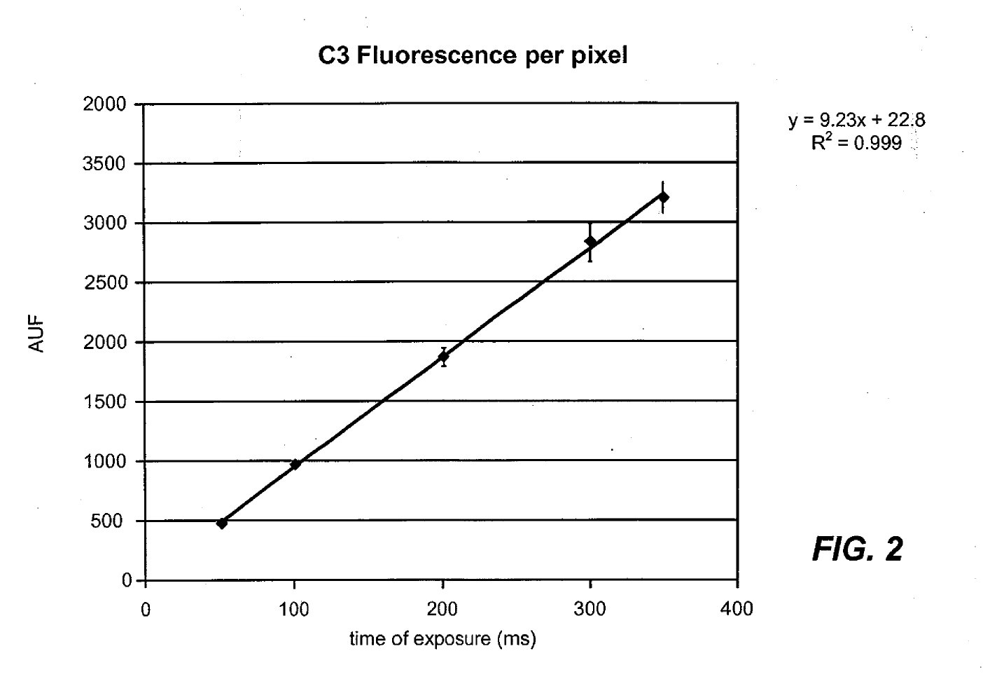

A fluorescence microscopy standard (4.0 rim-diameter microspheres, Kit M-7901 from Molecular Probes) was used to calibrate two different Leica microscopes. Standard curves of fluorescence per pixel (average of about 20 microspheres at each time point) versus exposure time in milliseconds were constructed (see FIG. 1 and FIG. 2). A linear response is observed with both microscopes over the range of exposure time used to acquire images.

A breast cancer cell line, HCC 2218 (positive for HER2/neu expression) was stained simultaneously with anti-cytokeratin-FITC and anti-HER2/neu-Alexa 532 (red fluorescence) antibodies. Digital images were acquired of identical fields using filter cubes that differentiate the two fluorescence signals. The HER2/neu images were acquired using exposure times within the linear range of the standard curves, viz., 500 milliseconds for microscope A (FIG. 1) and 300 milliseconds for microscope B (FIG. 2). The FITC signals are used to identify the breast cancer cells and to outline the spatial areas of interest; these outlines are saved.

The outlines are recalled and overlayed on the Alexa 532 image of an identical field of cells. The software (Image-Pro Plus) generates a table showing the area of each region of interest (ROI, determined from FITC fluorescence) and the mean fluorescence per pixel in each region (HER2/neu signal). The data from two slides examined on two different microscopes are presented in the table below.

| | Leica A | Leica B |
|---|---|---|
| Number of ROIs | 26 | 36 |
| | 23 | 28 |
| Mean Fluorescence | 837 | 673 |
| | 753 | 585 |
| Standard deviation | 204 | 215 |
| | 185 | 180 |
| Percent of Standard | 39.0 | 24.1 |
| | 35.1 | 21.0 |

In this experiment the digital values (expressed as the percent of our fluorescence standard at an equivalent exposure time) for the amount of HER2/neu protein for cells on the same slides and obtained on two different microscopes do not favorably compare in that the average value for the two slides is 1.64-fold higher for microscope A compared to microscope B. This difference may be due to the fact that the exposure times used to acquire the HER2/neu images do not give the same value for intensity when the same reference standard is used on the two microscopes. Further experiments showed that when the exposure times were adjusted to give the same intensity measurement for the reference standard on the two microscopes, the HER2/neu values were much more consistent, as discussed below.

### Example 2

It was then determined that the desired consistency of results could be achieved by the following method.

An antibody specific for the receptor is labeled with fluorescent dye and a fluorescence standard has been obtained for the generation of standard curves to allow HER2/neu fluorescence to be normalized as a percentage of the non-bleaching standard. HER2/neu values can then be correlated from sample to sample and from laboratory to laboratory based on quantitative calibration on a universal fluorescence standard.

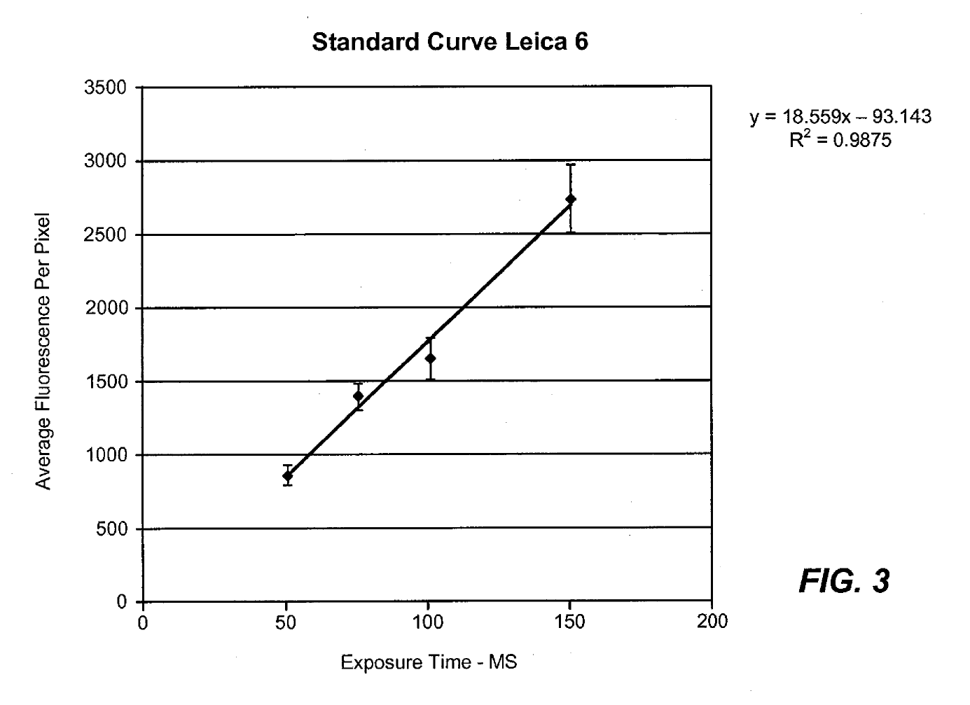

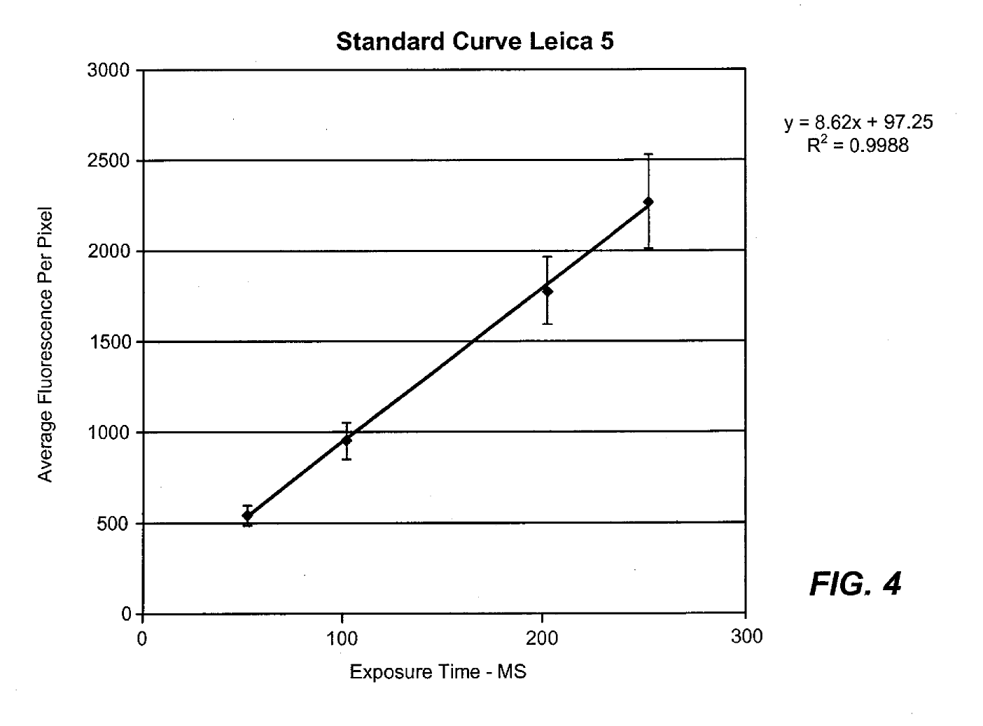

A fluorescence microscopy standard (4.0 um-diameter microspheres, Kit M-7901 from Molecular Probes) was used to calibrate two different Leica microscopes. Standard curves of fluorescence per pixel (average of about 20 microspheres at each time point) versus exposure time in milliseconds were constructed (see FIG. 3 and FIG. 4). A linear response is observed over the range of exposure time used to acquire images. From these linear curves, an exposure time that produces a fluorescence intensity per pixel of 2000 (approximately one half of the saturation value) was calculated and found to be 221 and 113 milliseconds for our Leica 5 and Leica 6 microscopes, respectively.

Two breast cancer cell lines, HCC 2218 (positive for HER2/neu expression) and HCC 38 (negative for HER2/neu expression) were stained simultaneously with anti-cytokeratin-FITC and anti-HER2/neu-Alexa 532 (red fluorescence). Digital images were acquired of identical fields using filter cubes that differentiate the two fluorescence signals. The FITC signals are used to identify the breast cancer cells and to outline the spatial areas of interest; these outlines are saved. The outlines are recalled and overlayed on the Alexa 532 image of an identical field of cells. The software (Image-Pro Plus) generates a table showing the area of each region of interest (ROT, determined from FITC fluorescence) and the mean fluorescence per pixel in each region (HER2/neu signal). The data from two slides examined on two different microscopes are presented in the tables below.

Leica 5

| | HCC 2218 | | HCC 38 | |
|---|---|---|---|---|
| | Sample 1 | Sample 2 | Sample 1 | Sample 2 |
| Number of | 23 | 35 | 22 | 20 |
| ROIs | | | | |
| Mean | 296 | 372 | 33 | 28 |
| Fluorescence | | | | |
| Standard | 110 | 111 | 12 | 10 |
| Deviation | | | | |
| Percent of | 14.8% | 18.6% | 1.7% | 1.4% |
| Standard | | | | |

Leica 6

| | HCC 2218 | | HCC 38 | |
|---|---|---|---|---|
| | Sample 1 | Sample 2 | Sample 1 | Sample 2 |
| Number of | 27 | 27 | 22 | 22 |
| ROIs | | | | |
| Mean | 280 | 284 | 24 | 26 |
| Fluorescence | | | | |
| Standard | 89 | 78 | 10 | 7 |
| Deviation | | | | |
| Percent of | 14.0% | 14.2% | 1.2% | 1.3% |
| Standard | | | | |

The digital values representing the amount of HER2/neu protein are approximately 10-fold higher for the cell line positive for expression (HCC 2218) compared to the cell line negative for expression (HCC 38) and the values between the two microscopes are consistent. The table presented above presents quantitative data on the HER-2/neu receptor obtained with two different microscopes using two cell lines, one positive for the receptor and one negative for the receptor. A standard curve was generated for each microscope and the calculated exposure times for image acquisition were 221 ms (Leica 5) and 113 ms (Leica 6). The data obtained with duplicate slides were very similar on the same microscope as well as on the two different microscopes. This demonstrates that the methods of the present invention for normalizing quantitative data to the same fluorescence standard can allow for comparison of daily fluorescence measurements and furthermore, can obtain very similar quantitative data on two different instruments.

### Example 3

Cells: Six breast cancer cell lines were purchased from ATCC and grown in medium containing 10% fetal bovine serum. HCC2218, HCC38, HCC2O2 and T-47D were grown in RPMI 1640, MCF-7 was grown in EMEM and SK-BR-3 was grown in McCoy's. Exponentially growing cells were trypsinized and spun onto microscope slides from a megafunnel with a Cytospin 3 (Shandon) at 1000 rpm for 10 minutes and then air-dried for at least two hours, preferably overnight.

Reagents: An antibody cocktail for identifying epithelial cells containing monoclonal antibodies covalently labeled with FITC and which recognizes nine different cytokeratin peptides and a tumor-associated glycoprotein. Anti-ERCC-1 (sc-10785, Santa Cruz Biotechnology), anti-thymidylate synthase (clone TS 106, Exalpha Biologicals), anti-estrogen receptor (clone TE 111 .5D 11, Exalpha Biologicals) and Trastuzumab (Genentech) were conjugated to Alexa Fluorescent (AF) dyes AF 594, AF 647, AF 594 and AF 532 (Molecular Probes, Eugene, Oreg.), respectively, using succinimidyl ester protein labeling kits (Molecular Probes). MULTISPECK fluorescence microscopy standard (kit M-7901, Molecular Probes) consisting of 4.0 micron-diameter, multispectral fluorescence microspheres was used to calibrate the microscope. Permeability buffer (2×) contains 1% BSA and 0.2% saponin in PBS. Vectashield mounting medium containing DAPI was purchased from Vector Laboratories.

Staining of cells: Air-dried cancer cells on microscope slides were fixed in 2% paraformaldehyde for 10 minutes at 4-8° C., washed in PBS for 10 minutes and blotted dry. Cells were incubated with the fluorescently-labeled antibodies in permeability buffer (1X) at 4° C. for 22 hours. Incubations contained the following: 1) epithelial cell staining cocktail (ECSC)-FITC and trastuzumab-AF 532 (HER-532, dye/protein=4.3) both at 5 μg/ml; 2) ECSC-FITC, HER-532, anti-ERCC-1-AF 594 (dye/protein=8) at 5 μg/ml and anti-thymidylate synthase-AF 647 at 20 μg/ml; or 3) ECSC-FITC, HER-532, anti-estrogen receptor-AF 594 at 10 μg/ml. After incubation, the cells were washed twice in PBS at RT, 5 minutes each wash. The slides were blotted dry and mounted with DAPI-containing medium under a coverslip.

Fluorescence microscopy: Stained cells were examined on a Leica DM RXA microscope equipped with a Princeton Instruments MicroMax Digital CCD Camera System (Model 1 300YHS) and filter cubes which allow for differentiation of five fluorescence signals. Excitation, dichroic and emission filters in each cube are for DAPI 360 nm/400 nm/470 nm, for FITC 470 nm/497 nm/522 nm, for AF 532 546 nm/557 nm/567 nm, for AF 594 581 nm/593 nm/617 nm, and for AF 647 630 nm/649 nm/667 nm. Images of stained cells were acquired with a 40× objective using Image-Pro Plus software.

Calibration of the microscopy system: In order to conduct quantitative immunofluorescence studies, one must be able to acquire digital images and compare fluorescence measurements obtained over various time periods and on different microscopes. This was accomplished by calibrating two Leica microscopy systems with a fluorescence standard which contained four micron-diameter fluorescent microspheres. A suspension of microspheres is placed on a microscope, air dried and mounted in medium under a coverslip. Images are acquired at various exposure times being sure not exceed times that result in a saturation level, i.e., 4096 fluorescence units per pixel. The images are processed in Image-Pro Plus to obtain the average fluorescence intensity per pixel of about 20 microspheres at each exposure time as follows: 1) the bitmap of each image contains the fluorescence intensity of each pixel in numeric units; 2) by binarizing the image one can determine a threshold that distinguishes the microspheres from the background; 3) selecting the appropriate size of the regions of interest and for the range of intensities allows one to outline/count the microspheres and to obtain the average fluorescence per pixel within the outline; 4) the measurement data can be viewed to eliminate microspheres on the fringe of the image and those outside the area of uniform illumination. The measurement data (area and average fluorescence/pixel) is saved and exported to MICROSOFT Excel (spreadsheet program) to calculate the mean fluorescence per pixel and standard deviation of about 20 microspheres at four to five different exposure times. A plot of mean fluorescence intensity against exposure time is generated to obtain the slope and intercept of the linear regression. The exposure time required to yield a value of 2000 fluorescence units (one half the saturation level) for the microscope and filter cube to be used for acquiring images of biomarker-stained cells is calculated. Thus by using the same fluorescence standard, each microscope can be calibrated to yield the same fluorescence intensity by selecting the appropriate exposure time. It is recommended that, when possible, exposure times be kept under one second to eliminate any photobleaching.

Quantifying of HER-2/neu receptor on breast cancer cells: Six different breast cancer cell lines were stained with ESCE-FITC and trastuzumab-AF 532. Images were obtained with the appropriate filter cubes; the exposure time used to acquire the AF 532 images was either 417 or 426 milliseconds as determined on the same day the cancer cells were examined. The FITC images (represent cytokeratin) were used to outline the spatial area of the cells. These outlines were saved and later recalled to be overlaid on an AF 532 image (represent HER-2/neu receptor) of an identical field of cells. An additional two outlines on the image were drawn in an area with no cellular fluorescence to get a measure of the background. The Image-Pro Plus software generates a table showing the area of each region of interest (ROT, cancer cell determined from cytokeratin fluorescence) and the average fluorescence per pixel for the ROI due to the trastuzumab-AF 532. The data is saved and exported to Microsoft Excel for processing, viz., subtract background and calculate the mean trastuzumab-AF 532 fluorescence of 20 to 40 cells. The average value thus obtained is normalized by dividing by 2000 and reported as a percentage of the fluorescence standard. This allows for a comparison of different experiments and different cell lines.

Characterization of breast cancer cells with multiple fluorescent biomarkers: In order to stain cells with multiple mouse monoclonal antibodies it is possible to directly label each antibody. In this study this was accomplished with fluorescein isothiocyanate and succinimidyl ester derivatives of AF 532, AF 594, AF 647 and the use of a DNA counter stain (DAPI). A breast cancer cell line (SK-BR-3) was incubated for 22 hours at 4° C. with the following four antibodies, each labeled with one of the above fluorescent derivatives: anti-estrogen receptor (ER), trastuzumab (HER-2), anti-thymidylate synthase (TS) and ECSC-FITC (CK) or anti-ERCC-1, trastuzumab, TS and ECSC-FITC. Images were acquired with a 40× objective using filter cubes that allow for discrimination of the five fluorescence signals in the same cell. Positive fluorescence signals can be seen in the same cell for the estrogen receptor (ER, AF 594), HER-2/neu receptor (HER-2, AF 532) and thymidylate synthase (TS, AF 647); positive signals for DAPI (nuclear DNA) and cytokeratin (CK) indicate the that signals are in a intact epithelial cell. Similar results are seen when anti-estrogen receptor is replaced by anti-ERCC-1. The presence of these biomarkers in circulating cancer cells can be used to predict the response to certain drugs when the biomarker is related to the mechanism of action of the drug. For example, one would predict that cells which have a high density of the HER-2/neu receptor would respond to treatment with trastuzumab or that a drug such as cisplatin would be less effective in cells with a high repair capacity which can be reflected by measurement of ERCC-1 (excision repair cross-complementary 1). The present invention has established a paradigm for the quantitative measurement of biomarkers in single cells using trastuzumab labeled with AF 532 to stain the HER-2/neu receptor in breast cancer cells as a model system. The data is presented in the following section.

Quantifying the HER-2/neu receptor in breast cancer cells: The first priority in conducting this quantitative immunofluorescence study was to establish a method for comparing daily fluorescence measurements. Leica fluorescence microscopes were calibrated using a fluorescence reference standard, which consisted of 4 micron-diameter microspheres. Other reference standards may be used to calibrate instruments to be used in the practice of the methods of the invention. The calibrations were performed using identical 40X objectives and filter cubes (excitation 546 nm/dichroic 557 nm/emission 567 nm). Exposure times were chosen to prevent reaching saturation levels. Standard curves were obtained that showed a linear relationship between the mean fluorescence intensity of the microspheres (about 20 at each time point) and exposure time. The slope and intercept were used to calculate the exposure time required to yield an average fluorescence intensity of 2000 with the reference standard. A new curve may be generated as desired, e.g., every week, and on each day an image is acquired and processed to ensure that the 426 ms exposure falls within 10% of 2000 fluorescence intensity units. Other fluorescent intensity levels and corresponding exposure times may be chosen and used in the practice of the invention. Typically a fluorescent intensity level and exposure time will be chosen so as to be in the linear response range of the instrument to be used for the analysis of the cell sample.

Six breast cancer cell lines were stained with trastuzumab-AF 532 and ECSC-FITC. Images were acquired at the appropriate exposure time (that which yields a value of 2000 with the standard) and analyzed to determine the average fluorescence per pixel in each ROT (cancer cell) imaged with the C3 filter cube (AF 532 signal). The spatial area of each ROT was determined from the cytokeratin fluorescence which is very strong. The outlines are saved, recalled and overlaid on an AF 532 image of an identical field of cells. The software generates a table showing the area and average fluorescence per pixel of each ROT.

The following represents quantitative data from duplicate slides for the amount of HER-2/neu receptor on six breast cancer cell lines when stained with an identical antibody preparation. Each value represents the mean of 20 to 40 cancer cells.

Percent of Standard Fluorescent Microspheres*

| Cell Line | Slide #1 | Slide #2 | Average |
|---|---|---|---|
| HCC 2218 | 57.2 | 46.1 | 51.6 |
| SK-BR-3 | 20.9 | 20.6 | 20.8 |
| HCC 202 | 3.2 | 4.2 | 3.7 |
| HCC 38 | 3.7 | 4.3 | 4.0 |
| MCF-7 | 3.2 | 2.9 | 3.0 |
| T47D | 3.4 | 3.5 | 3.4 |

The data generated from duplicate slides are quite comparable. Two of the cell lines show an overexpression of the receptor while the other four show only background fluorescence. The data is presented as a percentage of the fluorescence standard microspheres using an exposure time 426 ms for this experiment as determined from the standard curve. It is predicted that two of the cell lines (HCC 2218 and SK-BR-3) would be more susceptible to trastuzumab than the other four.

While the above examples measure fluorescence per pixel, other techniques could be used to measure the presence, absence and/or amount of a biomarker (for example, fluorescence (or other reporter) per cell or per other area of interest).

Using this technique, and correlating the result with clinical data, a threshold value may be determined for a particular therapy against a particular disease.

While illustrated with respect to the HER2/neu test for trastuzumab therapy, this is only one of a class of compounds where the likely therapeutic benefit of the therapy depends on whether or not the patient is overexpressing or underexpressing a protein or other biological biomarker, and the invention may be applied to any similar therapy using the same techniques, in a manner which would be known to one skilled in the art.

Therefore, while a specific embodiment of the invention has been shown and described in detail to illustrate the application of the principles of the invention, it will be understood that the invention may be embodied otherwise without departing from such principles and that various modifications, alternate constructions, and equivalents will occur to those skilled in the art given the benefit of this disclosure. Although the foregoing refers to particular embodiments, it will be understood that the present invention is not so limited. It will occur to those of ordinary skill in the art that various modifications may be made to the disclosed embodiments and that such modifications are intended to be within the scope of the present invention. All patents, patent applications and publications cited herein are fully incorporated by reference.

1. A method for predicting efficacy of one or more therapeutic agents, comprising:
obtaining one or more cell samples from a subject at one or more time points, wherein the one or more samples comprise cancer cells and the cancer cells comprise one or more biomarkers associated with the one or more therapeutic agents;
contacting the one or more cell samples with one more binding moieties that specifically bind the cancer cells through one or more biomarkers, wherein the one or more binding moieties further comprise a fluorescent moiety;
measuring fluorescence intensity of the one or more biomarkers on one or more cancer cells with a microscopy system;
quantifying the expression levels of the one or more biomarkers, thereby predicting the efficacy of the one or more therapeutic agents from a group consisting of gefitinib, cetuximab, trastuzumab, gemcitabine, cisplatinum, paclitaxel, vinorelbine, 5 FU-related drugs, and bevacizumab.

2. The method of claim 1, wherein the cells comprise the one or more biomarkers.

3. The method of claim 1, wherein the one or more biomarkers comprise epidermal growth factor receptor (EGFR), Her2/neu receptor (Her2), ribonuclease reductase, excision repair cross complementary 1 (ERCC-1), beta tubulin III, vascular endothelial cell growth factor (VEGF), or any combination thereof.

4. The method of claim 3, wherein the one or more biomarkers comprise cytokeratin.

5. The method of claim 3, wherein the one or more biomarkers comprise EGFR.

6. The method of claim 1, wherein the one or more samples is obtained from a body fluid.

7. The method of claim 6, wherein the body fluid is blood.

8. The method of claim 1, wherein quantifying the expression levels of the one or more biomarkers comprises analysis of the fluorescent intensity of the one or more biomarkers.

9. The method of claim 8, wherein quantifying the expression levels of the one or more biomarkers comprises comparing the fluorescent intensity of the one or more biomarkers to a fluorescent intensity of one or more reference standards comprising fluorescent microspheres or fluorescent beads.

10. The method of claim 1, wherein the one or more time points comprise one or more time points before, during, or after administration of the one or more therapeutic agents to a subject.

11. The method of claim 1, wherein predicting the efficacy of the one or more therapeutic agents comprises comparing the fluorescent intensity measurements of samples obtained from a subject at two or more time points.

12. The method of claim 11, wherein an increase or no change in the fluorescent intensity measurements determined from a sample obtained at one or more time points after a sample obtained at one or more other time points is indicative of a negative response to the one or more therapeutic agents.

13. The method of claim 11, wherein a decrease in the fluorescent intensity measurements determined from a sample obtained at one or more time points after a sample obtained at one or more other time points is indicative of a positive response to the one or more therapeutic agents.

14. The method of claim 1, wherein the one or more binding moieties comprise one or more antibodies.

15. The method of claim 14, wherein the one or more antibodies comprise gefitinib, cetuximab, trastuzumab, and bevacizumab.
